(19) United States
(12) Patent Application Publication
(10) Pub. No.: US 2024/0197210 A1
(43) Pub. Date: Jun. 20, 2024

(54) Method for Measuring the Plasma Concentration of Medication in a Human Patient Through the Use of Continuous Glucose Monitors

(71) Applicant: Matthew Lucci, Venus, TX (US)

(72) Inventor: Matthew Lucci, Venus, TX (US)

(73) Assignee: The Runatek Corporation, Dallas, TX (US)

(21) Appl. No.: 18/068,508

(22) Filed: Dec. 19, 2022

(51) Int. Cl. A61B 5/145; A61M 5/172

(52) U.S. Cl. CPC A61B 5/14532; A61M 5/1723

(57) ABSTRACT

This disclosure relates to a method for determining the presence of acetaminophen in a human patient through the use of continuous glucose monitors, such as continuous analyte sensors. One of the sensors is subject to the distortion caused by acetaminophen interference with the sensor's oxidation reaction, and one of the sensors is not impacted by such distortion, allowing for a measurement of the difference between the glucose measurements of these two sensors. This disclosure also relates to a method for determining the concentration of medications co-administered or combined with acetaminophen in a human patient through similar means.

### REFERENCES CITED

US PATENT DOCUMENTS

| | | | |
|---|---|---|---|
| 7,657,297 | B2 | February 2010 | Simpson |
| 8,456,301 | B2 | June 2013 | Fennell |
| 8,612,159 | B2 | December 2013 | Say |
| 9,037,210 | B2 | May 2015 | Simpson |
| 9,750,441 | B2 | September 2017 | Brauker |
| 10,251,605 | B2 | April 2019 | Liu |
| 10,327,684 | B2 | June 2019 | Park |
| 11,064,917 | B2 | July 2021 | Simpson |
| 2022033609 | A1 | October 2022 | Sanigepalli |
| 63/291,455 | | December 2021 | Lucci |

### OTHER PUBLICATIONS

Basu, A., et al., “Continuous Glucose Monitor Interference with Commonly Prescribed Medications: a Pilot Study,” Journal of Diabetes Science and Technology, vol. 11(5), 2017, 936-941.
Basu, A., et al., “Direct Evidence of Acetaminophen Interference with Subcutaneous Glucose Sensing in Humans: A Pilot Study,” Diabetes Technology & Therapeutics, vol. 18(Suppl 2), 2018; S243-S247.
Calhoun P., et al., “Resistance to Acetaminophen Interference in a Novel Continuous Glucose Monitoring System,” Journal of Diabetes Science and Technology, vol. 12(2), 2018, 393-396.
Devarakonda K., et al., “Single- and multiple-dose pharmacokinetics of biphasic immediate-release/extended-release hydrocodone bitartrate/acetaminophen (MNK-155) compared with immediate-release hydrocodone bitartrate/ibuprofen and immediate-release tramadol HCl/acetaminophen,” Journal of Pain Research, vol. 8, 2015, 647-656.
Lucci M., et al., “Development of a Mathematical Model for Detecting Hydrocodone Concentrations in Interstitial Fluid with Continuous Glucose Monitors,” 16 Dec. 2022, The Runatek Corporation. https://www.runatek.com/_files/ugd/dfc27c_3 f417bf8a1344f10ab8f2aee061f3477.pdf
Maahs D. M., et al., “Effect of Acetaminophen on CGM Glucose in an Outpatient Setting,” Diabetes Care, vol. 38(10), 2015, e158-e159.
McGarraugh G., “The Chemistry of Commercial Continuous Glucose Monitors” Diabetes Technology & Therapeutics, vol. 11(Supplemental), 2009, S17-24.
Moatti-Sirat D., et al., “Evaluating In Vitro and In Vivo the Interference of Ascorbate and Acetaminophen on Glucose Detection by a Needle-Type Glucose Sensor,” Biosensors and Bioelectronics, vol. 7, 1992, 345-352.
Moatti-Sirat D., et al., “Reduction of Acetaminophen Interference in Glucose Sensors by a Composite Nafion Membrane: Demonstration in Rats and Man,” Diabetologia, vol. 37(6), 1994, 610-616.
Monsod T. P., et al., “Do Sensor Glucose Levels Accurately Predict Plasma Glucose Concentrations During Hypoglycemia and Hyperinsulinemia?,” Diabetes Care, vol. 25 (5), 2002, 889-893.
Palmisano F., et al., “Simultaneous Monitoring of Glucose and Lactate by an Interference and Cross-Talk Free Dual Electrode Amperometric Biosensor Based on Electropolymerized Thin Films,” Biosensors & Bioelectronics, vol. 15, 2000, 531-539.
Schiavon M., et al., “A Model of Acetaminophen Pharmacokinetics and its Effect on Continuous Glucose Monitoring Sensor Measurements.” Annual International Conference of the IEEE Engineering in Medicine and Biology Society, 2018; 1:159-162.
Singla A., et al., “Pharmacokinetic evaluation of hydrocodone/acetaminophen for pain management,” Journal of Opioid Management, vol. 9(1), 2013, 71-80.
Tierney M. J., et al., “Effect of Acetaminophen on the Accuracy of Glucose Measurements Obtained with the GlucoWatch Biographer,” Diabetes Technology & Therapeutics, vol. 2(2), 2000, 199-207.
Yue Y, et al., “Selection of 12-Hour Sustained-Release Acetaminophen (Paracetamol) Formulation Through Comparison of Pharmacokinetic Profiles of 4 Sustained-Release Prototype Formulations and Standard Acetaminophen Formulation,” Clinical Pharmacology in Drug Development, vol. 7(12), 2017, 87-94.
Zhang, et al., “Elimination of the Acetaminophen Interference in an Implantable Glucose Sensor,” Analytical Chemistry, vol. 66(7), 1994, 1183-1188.

### CROSS-REFERENCE TO RELATED APPLICATIONS

This application claims priority to the U.S. Provisional Patent Application No. 63/291,455 filed Dec. 19, 2021 in the name of Lucci entitled Method for Measuring the Plasma Concentration of Medication in a Human Patient Through the Use of Continuous Glucose Monitors which is hereby incorporated by reference in its entirety.

### TECHNICAL FIELD

The disclosure herein relates to a method for measuring the plasma concentration of medication in a patient's system through a glucose oxidation reaction, such as through the use of continuous glucose monitors. This disclosure is directed to sensing of prescription medications through the use of continuous analyte sensors.

### BACKGROUND

It is known that acetaminophen is an interfering species for existing continuous analyte sensors and that acetaminophen can interfere with readings of a continuous glucose monitoring (CGM) system, leading to erroneously high glucose readings. This has long been a challenge for CGM systems. CGM monitoring systems work by measuring the glucose concentration in the subcutaneous tissue by utilizing a needle electrode to exploit the glucose-oxidase reaction as described by McGarraugh in The Chemistry of Commercial Continuous Glucose Monitors: Diabetes Technology Therapeutics, vol. 11, 2009.

Acetaminophen does not cause significant interference with glucometers or glucose meters, such as typical finger-prick glucose meters. However, the presence of acetaminophen in a patient's system causes a distortion of the patient's continuous glucose monitor readings because of its interference with such reaction, as explained by Basu et al. in Continuous Glucose Monitor Interference with Commonly Prescribed Medications: a Pilot Study: Journal of Diabetes Science and Technology, vol. 11, 2017, and further described in Schiavon et al.'s A Model of Acetaminophen Pharmacokinetics and its Effect on Continuous Glucose Monitoring Sensor Measurements: Annual International Conference of the IEEE Engineering in Medicine and Biology Society, 2018.

While this has long been considered a flaw in CGM technology, the advent of CGM sensors that are impervious to such interference opens the possibility for this phenomenon to be utilized for medication detection. The effect can be measured in real time through a comparison with an accurate benchmark. Every CGM device type and series displays a different measurement distortion response to the presence of acetaminophen. The acetaminophen-dependent measurement distortion for CGM device types and series can be modeled mathematically and by computer. Such technology also opens the possibility of this effect being harnessed for medication monitoring.

Several medications also include acetaminophen as a component or ingredient, including in combination with a high-strength opioid pain reliever, as discussed in Singla et al.'s Pharmacokinetic evaluation of hydrocodone/acetaminophen for pain management: Journal of Opioid Management, vol. 9, no. 1, 2013.

Acetaminophen is also commonly dosed with other medications in a method referred to herein as co-administration. This method involves the use of multiple medications to treat an ailment, wherein the medications are not combined into a single drug.

Elevated concentrations of acetaminophen have been shown to have negative effects on patients, including liver failure, as described by McGill et al. in The mechanism underlying acetaminophen-induced hepatotoxicity in humans and mice involves mitochondrial damage and nuclear DNA fragmentation: The Journal of Clinical Investigation, vol. 122, no. 4, 2012. All of the above references are hereby incorporated by reference in their entirety.

Elevated or insufficiently low concentrations of combination medications or medications that are co-administered with acetaminophen also lead to harmful effects on patients. In patients being treated with acetaminophen in combination with opioid medications, elevated concentrations of opioid pain medications can cause a dependency or addiction to develop. In other types of medications, adverse effects from elevated or insufficient concentrations are common.

Combination medications and medications that are co-administered with acetaminophen have thus far proven difficult to measure, particularly through means of continuous monitoring. The current state of the art includes lab tests for each medication that usually involve drawing significant quantities of blood to test for the plasma concentration of a given medication present at the time that the sample was taken. Therefore, it is recognized that the development of a minimally invasive process for measuring the concentration of medications in real time may be beneficial to both a patient and the treating physician.

### SUMMARY OF THE INVENTION

One embodiment of the invention is a process for determining the blood plasma concentration of acetaminophen in a patient's system by measuring the effect of the acetaminophen's distortion of blood glucose readings through an oxidation reaction, comprising (a) an accurate, non-distorted reading of the plasma glucose concentration, and (b) a continuous glucose monitor (CGM) with a consistent and observable distortion due to acetaminophen interference, and (c) a computer algorithm that utilizes the values and difference in values measured from (a) and (b) as inputs to determine the concentration of acetaminophen present in the patient's blood plasma.

Another embodiment of the invention is a process for determining the blood plasma concentration of acetaminophen in a patent's system by measuring the effect of the acetaminophen's distortion of blood glucose readings, comprising (a) a CGM with a consistent and observable distortion due to acetaminophen interference, and (b) a separate CGM with a consistent and observable distortion due to acetaminophen interference that produces plasma glucose readings that are consistently and observably different from those produced by (a), and (c) a computer algorithm that utilizes the values and difference in values measured from (a) and (b) as inputs to determine the concentration of acetaminophen present in the patient's blood plasma.

Another embodiment of the invention is a process for determining the blood plasma concentration of medications containing acetaminophen in a patient's system by measuring the effect of the acetaminophen's distortion of blood glucose readings, comprising (a) an accurate, non-distorted reading of the plasma glucose concentration, and (b) a CGM with a consistent and observable distortion due to acetaminophen interference, and (c) a computer algorithm that utilizes the values and difference in values measured from (a) and (b) as inputs to determine the concentration of acetaminophen present in the patient's blood plasma, and (d) an algorithm that utilizes (c) as an input to determine the corresponding concentration of the medication containing acetaminophen.

An additional embodiment of the invention is a process for determining the blood plasma concentration of medications containing acetaminophen in a patent's system by measuring the effect of the acetaminophen's distortion of blood glucose readings, comprising (a) a CGM with a consistent and observable distortion due to acetaminophen interference, and (b) a separate CGM with a consistent and observable distortion due to acetaminophen interference that produces plasma glucose readings that are consistently and observably different from those produced by (a), and (c) a computer algorithm that utilizes the values and difference in values measured from (a) and (b) as inputs to determine the concentration of acetaminophen present in the patient's blood plasma, and (d) an algorithm that utilizes (c) as an input to determine the corresponding concentration of the medication containing acetaminophen.

A further embodiment of the invention is a process for determining the blood plasma concentration of certain medications that are co-administered with acetaminophen by measuring the effect of the acetaminophen's distortion of blood glucose readings, comprising (a) an accurate, non-distorted reading of the plasma glucose concentration, and (b) a CGM with a consistent and observable distortion due to acetaminophen interference, and (c) a computer algorithm that utilizes the values and difference in values measured from (a) and (b) as inputs to determine the concentration of acetaminophen present in the patient's blood plasma, and (d) an algorithm that utilizes (c) as an input to determine the corresponding concentration of the medication administered in conjunction with acetaminophen.

A further embodiment of the invention is a process for determining the blood plasma concentration of certain medications that are co-administered with acetaminophen by measuring the effect of the acetaminophen's distortion of blood glucose readings, comprising a) a CGM with a consistent and observable distortion due to acetaminophen interference, and (b) a separate CGM with a consistent and observable distortion due to acetaminophen interference that produces plasma glucose readings that are consistently and observably different from those produced by (a), and (c) a computer algorithm that utilizes the values and the difference in values measured from (a) and (b) as inputs to determine the concentration of acetaminophen present in the patient's blood plasma, and (d) an algorithm that utilizes (c) as an input to determine the corresponding concentration of the medication containing acetaminophen.

### BRIEF DESCRIPTION OF THE DRAWINGS

FIG. 1 is a graphical representation of the process of determining the concentration of co-administered medications in a human patient's blood, plasma, or interstitial fluid.

FIG. 2 is a graphical representation of the disclosed method of determining the plasma concentration of acetaminophen in a human patient using a glucometer for an accurate reference glucose concentration.

FIG. 3 is a graphical representation of the disclosed method of determining the plasma concentration of acetaminophen in a human patient using a continuous glucose monitor or continuous analyte sensor for an accurate reference glucose concentration.

FIG. 4 is a graphical representation of the disclosed method of determining the plasma concentration of medications co-administered with acetaminophen.

FIG. 5 is a cutaway view of dermal tissue with two continuous glucose monitors or continuous analyte sensors inserted into the dermis.

FIG. 6 is a cutaway view of dermal tissue with one continuous glucose monitor or continuous analyte sensor inserted into the dermis and one needle puncturing a capillary to draw blood for a glucometer measurement.

FIG. 7 is an in vivo measurement of interstitial glucose with two continuous glucose monitors in a human patient.

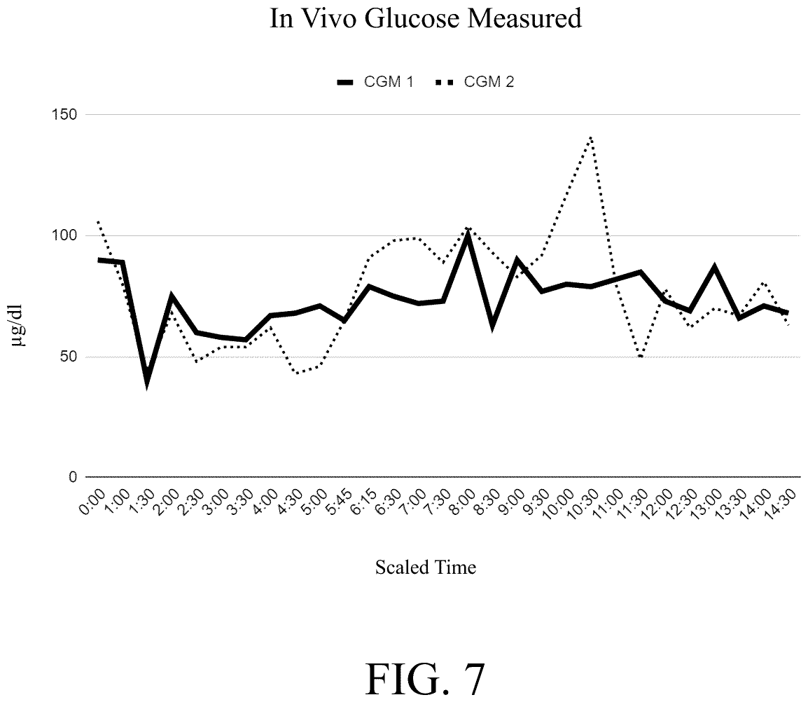

FIG. 8 is an in vivo measurement of interstitial glucose with one continuous glucose monitor compared with the blood glucose measured with an external glucometer.

FIG. 9 is an in vivo measurement of plasma acetaminophen concentrations using the disclosed method.

### DETAILED DESCRIPTION OF THE INVENTION

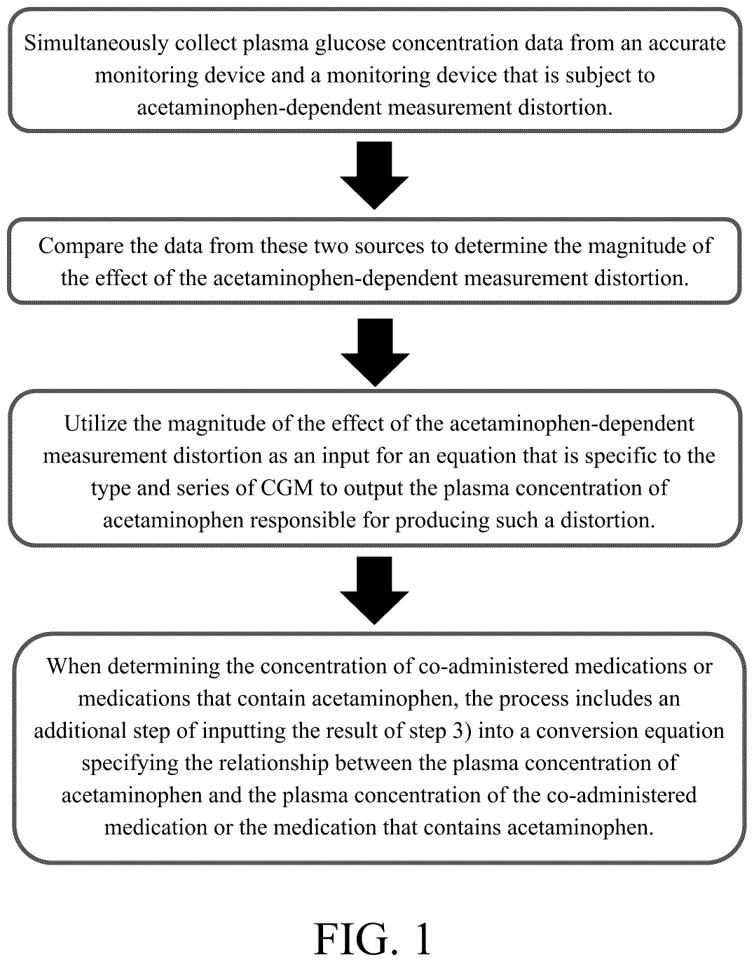

One embodiment of the present invention is directed to a process of determining the blood plasma concentration of acetaminophen by measuring the effect of CGM distortion due to the presence of acetaminophen in a patient's system by utilizing an accurate plasma glucose reading and a CGM with a consistent and observable distortion due to acetaminophen interference. As shown in FIG. 1, the process utilizes the plasma glucose readings from these two measurement methods as inputs in an algorithm that outputs the concentration of acetaminophen present in the blood plasma that produces such a distortion.

An accurate measurement of the plasma glucose, such as that from a glucometer, can be used as a baseline by which to compare the distorted plasma glucose reading from the CGM, and therefore determine the magnitude of the effect of the plasma concentration of acetaminophen. An alternative process is to utilize a second CGM that is not subject to significant glucose reading distortion due to acetaminophen in place of a glucometer.

CGMs work by measuring the glucose concentration in the subcutaneous tissue by utilizing a needle electrode to exploit the glucose-oxidase reaction as a means for determining the concentration of glucose in the blood plasma. The presence of acetaminophen in a patient's system can cause an interference with the patient's glucose monitor readings because of its interference with such reaction, leading to a consistent, repeatable, and observable distortion of the measurements collected by a CGM that is dependent upon the concentration of acetaminophen present in the blood plasma. The present invention utilizes a measurement of this phenomenon to determine the concentration of acetaminophen present in the blood plasma in real time.

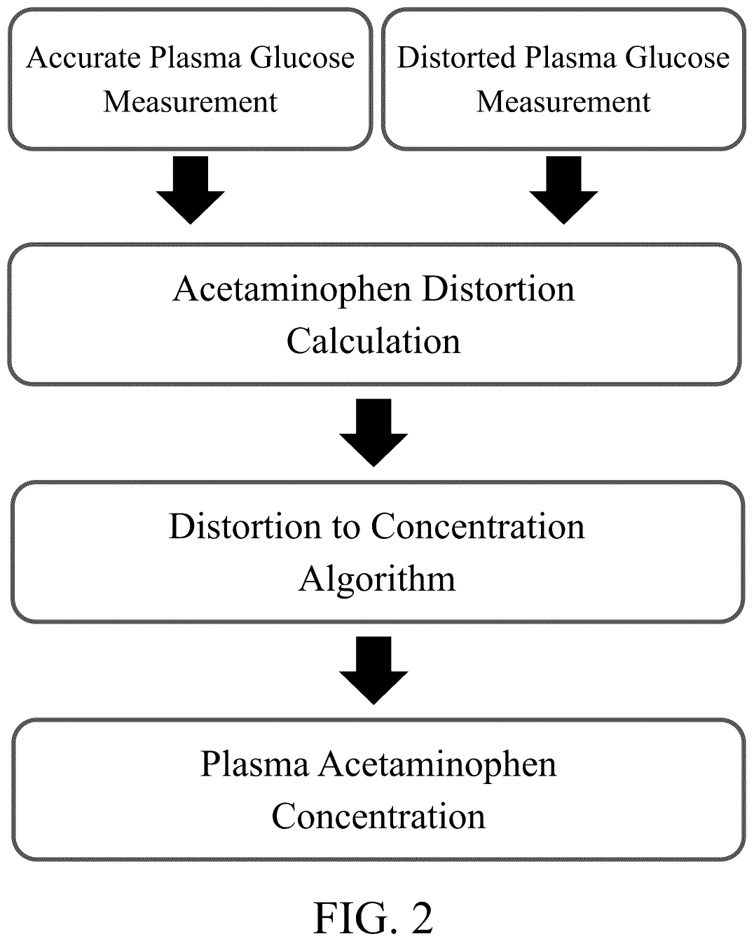

The measurement distortion can be used to determine the concentration of acetaminophen present in the blood plasma through the process outlined in FIG. 2. As shown, the process involves the collection of plasma glucose concentration data from an accurate glucose monitoring device and a glucose monitoring device that is subject to acetaminophen-dependent measurement distortion. The difference between these two readings at any point in time is the magnitude of the acetaminophen-dependent measurement distortion. Such distortion is then used to determine the plasma concentration of acetaminophen by using a mathematical or computer model of the effect of acetaminophen on the measurements of the CGM that is subject to distortion.

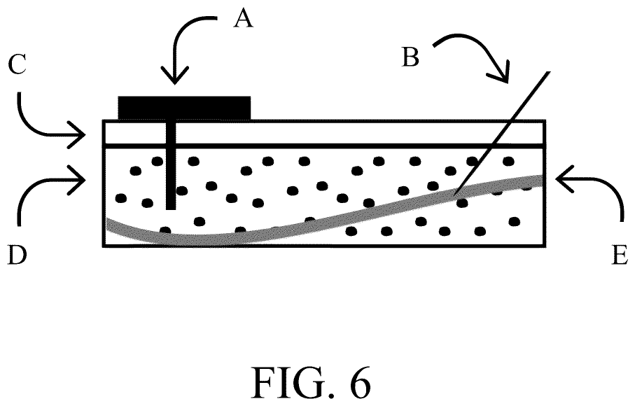

The present embodiment utilizes a CGM and an external glucometer, as shown in FIG. 6. In this embodiment, a continuous analyte sensor (A) is inserted into the dermis (D) and one needle (B) punctures a capillary (E) to draw blood for a glucometer measurement. The top layer of the skin is denoted as (C).

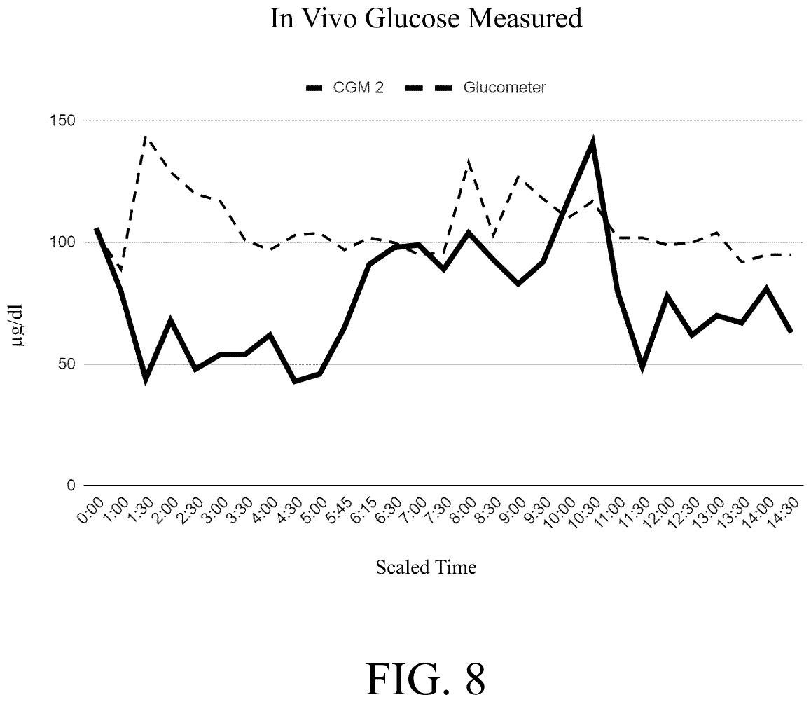

An example of this distortion effect is shown in FIG. 8, where the graph represents data from an in vivo measurement of interstitial glucose with one continuous glucose monitor compared with the blood glucose measured with an external glucometer. The CGM is subject to a distortion in measurement due to the acetaminophen oxidation reaction.

Another embodiment of the present invention is directed to a process of determining the concentration of acetaminophen in the interstitial fluid by measuring the effect of CGM distortion due to the presence of acetaminophen in a patient's system by utilizing an accurate plasma glucose reading and a CGM with a consistent and observable distortion due to acetaminophen interference. As shown in FIG. 1, the process utilizes the glucose readings from these two measurement methods as inputs in an algorithm that outputs the concentration of acetaminophen present in the interstitial fluid that produces such a distortion.

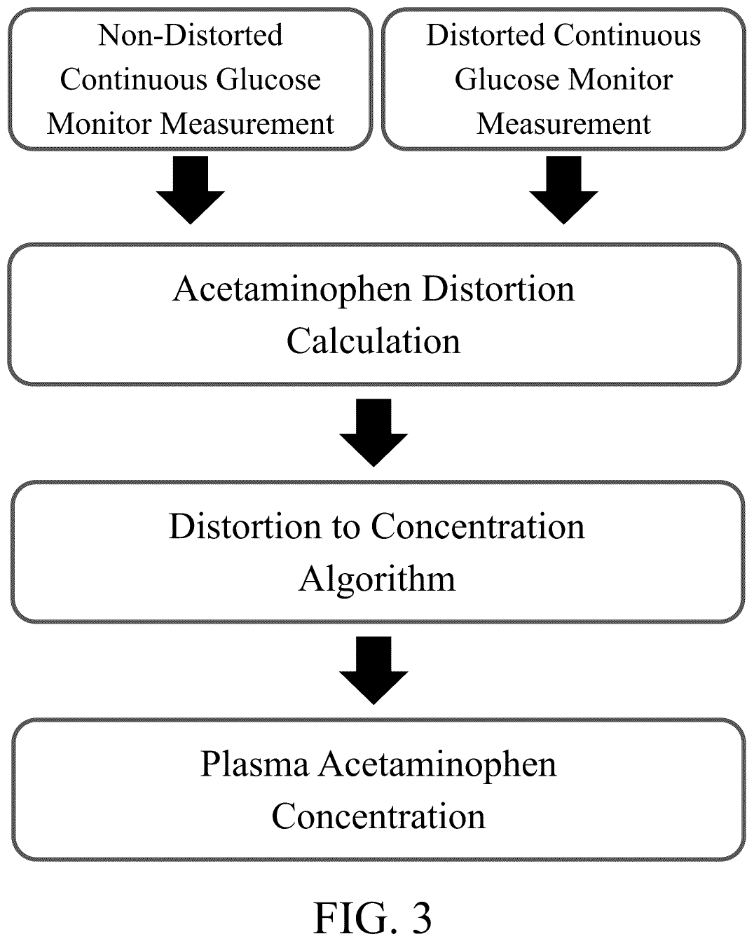

Another embodiment of the present invention is directed to a process of determining the blood plasma concentration of acetaminophen by measuring the effect of CGM distortion due to the presence of acetaminophen in the patient's system by utilizing two CGMs. The process uses two continuous glucose monitoring sensors to collect blood glucose levels from the patient. The readings from one of these sensors are subject to distortion due to the effects of acetaminophen in the patient's system, while the other is not. The difference between these two readings is used as an input in a distortion algorithm to determine the quantity of acetaminophen in the patient's system. As shown in FIG. 3, the process utilizes the plasma glucose readings from these two CGMs as inputs in an algorithm that outputs the concentration of acetaminophen present in the blood plasma that is responsible for producing such a distortion in real time.

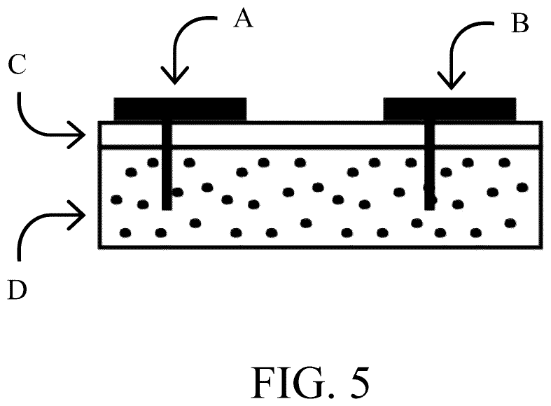

The present embodiment utilizes a set of two or more continuous glucose monitors, as shown in FIG. 5. These continuous glucose monitors (A) and (B) or continuous analyte sensors inserted into the dermis (D), where (A) is reasonably impervious to acetaminophen distortion and (B) is subject to acetaminophen distortion. The top layer of the skin is denoted as (C).

Both CGMs are subject to measurement distortion due to the presence of acetaminophen in the blood plasma. A repeatable, measurable difference in distortion between the two CGMs can be used to determine the concentration of acetaminophen in the blood plasma when the difference is dependent upon the concentration of acetaminophen present in the blood plasma. Acetaminophen-dependent measurement distortion can be measured by comparing data from two CGMs with or without a glucometer to serve as a baseline.

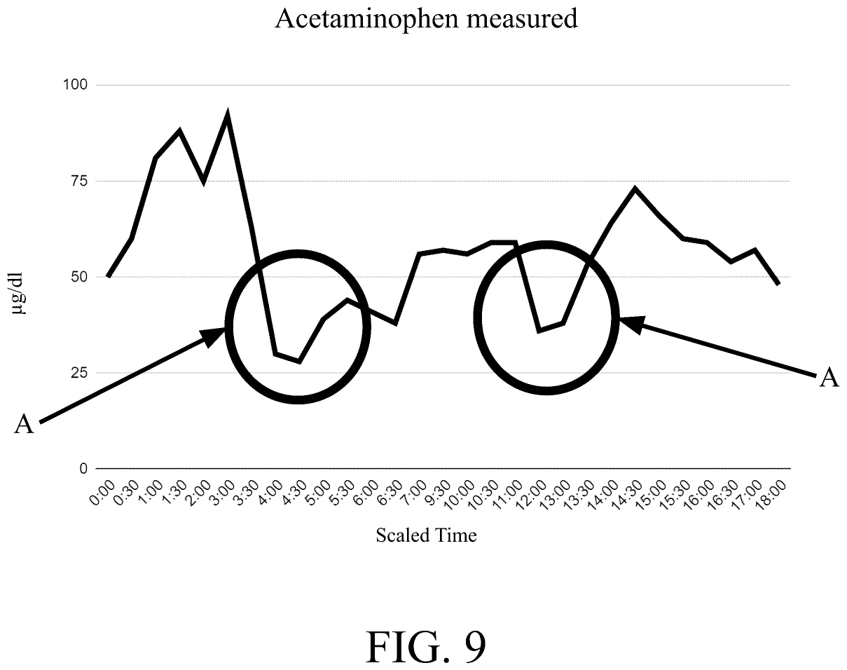

The detection of acetaminophen is demonstrated in FIG. 9 as an in vivo measurement of plasma acetaminophen concentrations using the disclosed method. The regions of the graph denoted with A are inflection points or local minima, and correspond to the intake of a dose of acetaminophen. The disclosed method of determining the presence of acetaminophen in a patient's blood, plasma, or interstitial fluid includes a temporal delay between the intake of the medication and its presence in the test fluid. A corrective algorithm may be used to process this data output and is considered to be a part of the present embodiment of the disclosed invention.

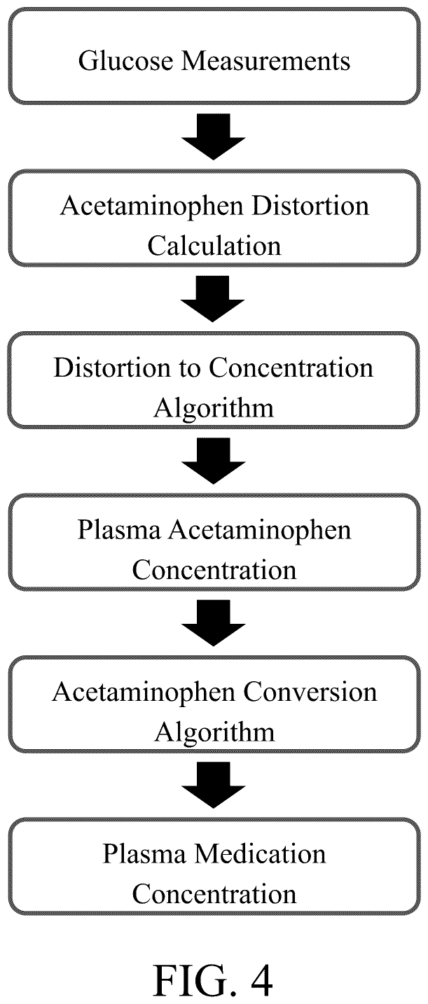

In one embodiment, the blood plasma concentration of medications that contain acetaminophen can be determined by an algorithm that converts the blood plasma concentration of acetaminophen to the blood plasma concentration of the medication that contains acetaminophen. When determining the concentration of medications that contain acetaminophen, the process includes an additional step of inputting the calculated acetaminophen concentration into a conversion equation specifying the relationship between the plasma concentration of acetaminophen and the plasma concentration of the medication that contains acetaminophen, as shown in FIG. 4.

In another embodiment, the process includes the conversion of the plasma concentration of acetaminophen to the plasma concentration of a medication containing acetaminophen. Certain medications are used in combination with acetaminophen in a set ratio. With this ratio, the process involves utilizing the output from the distortion algorithm as an input into two decay algorithms that account for time passed and quantity of doses of the combined medications. The process accounts for the ratio of two or more medications dosed in combination with acetaminophen to determine the plasma concentration of such medications in real time.

The blood plasma concentration of medications co-administered with acetaminophen can be determined by an algorithm that converts the acetaminophen concentration to the plasma concentration of the co-administered medication. This method includes the use of a mathematical or computer model of the disposition and decay behavior of the aforementioned medication to determine the relationship between the acetaminophen and the aforementioned medication in the blood plasma. Such a mathematical model is also referred to as the decay algorithm. The disclosed invention provides an output from the decay algorithm as the plasma concentration of the medication present in the patient's bloodstream at a given time

A further embodiment of the invention involves the use of CGM data to determine the plasma concentration of acetaminophen. Each type and series of CGM exhibits a different distortion effect due to the presence of acetaminophen. A key step in this embodiment is the determination of the magnitude and behavior of the distortion effect due to the plasma concentration of acetaminophen. This is achieved by taking glucose readings over time with a CGM and with a calibrated glucometer. These readings are compared against one another and considered with lab tested plasma glucose concentrations taken over the same time through the use of an artificial neural network to model the distortion relationship between acetaminophen and the associated CGM data when compared to the accurate glucose data.

The CGM data from two separate CGMs can be used to determine the plasma concentration. The individual CGMs are compared with a calibrated glucometer to determine the acetaminophen-dependent distortion for such model and series. Any CGM data can be measured against data obtained through a calibrated CGM or a CGM with a pre-determined acetaminophen-dependent distortion effect.

All publications and patent applications cited in this specification are hereby incorporated by reference as if each individual publication or patent application were specifically and individually indicated to be incorporated by reference. Although the foregoing has been described in some detail by way of illustration and example for purposes of clarity of understanding, it will be readily apparent to those skilled in the art in light of the teachings of this specification that certain changes and modifications may be made thereto without departing from the scope of the appended claims.

What is claimed is:

1. A method of determining the plasma concentration of acetaminophen by measuring the effect of the acetaminophen's distortion of blood glucose readings through continuous analyte sensors, comprising (a) an accurate, non-distorted reading of the plasma glucose concentration, and (b) a continuous glucose monitor (CGM) with a consistent and observable distortion due to acetaminophen interference, and (c) a computer algorithm that utilizes the values and difference in values measured from (a) and (b) as inputs to determine the concentration of acetaminophen present in the patient's blood plasma.

2. The method according to claim 1, wherein the non-distorted reading of the plasma glucose in (a) is measured with a glucose meter.

3. The method according to claim 1, wherein the non-distorted reading of the plasma glucose in (a) is measured with a Continuous Glucose Monitor without significant distortion.

4. The method according to claim 1, wherein the non-distorted reading of the plasma glucose in (a) is measured with a Continuous Glucose Monitor with a known and modeled distortion dependent upon the plasma concentration of acetaminophen.

5. A method according to claim 1, wherein the plasma concentration of medications that include acetaminophen as an ingredient are determined with a conversion algorithm from the determined plasma acetaminophen concentration.

6. A method according to claim 1, wherein the plasma concentration of medications that are coadministered with acetaminophen are determined with a conversion algorithm from the determined plasma acetaminophen concentration.

7. A method according to claim 1 wherein the presence of acetaminophen in a patient is detected through the determination of the existence of the distortion effect of acetaminophen on a continuous analyte sensor.

8. A method according to claim 1 wherein the presence of medications coadministered with acetaminophen is detected through the determination of the existence of the distortion effect of acetaminophen on a continuous analyte sensor.

9. A method according to claim 1 wherein the presence of acetaminophen in a patient is detected through the measurement of the distortion effect of acetaminophen on a continuous analyte sensor.

10. A method according to claim 1 wherein the presence of medications coadministered with acetaminophen is detected through the measurement of the distortion effect of acetaminophen on a continuous analyte sensor.

11. A method according to claim 1 wherein multiple continuous glucose monitors are used to determine the acetaminophen concentration.

12. A method according to claim 1 wherein multiple continuous analyte sensors are used to determine the acetaminophen concentration.

13. A method for determining the blood plasma concentration of acetaminophen in a patent's system, comprising (a) a CGM with a consistent and observable distortion due to acetaminophen interference, and (b) a separate CGM with a consistent and observable distortion due to acetaminophen interference that produces plasma glucose readings that are consistently and observably different from those produced by (a), and (c) a computer algorithm that utilizes the values and difference in values measured from (a) and (b) as inputs to determine the concentration of acetaminophen present in the patient's blood plasma.

14. A method according to claim 13, wherein the plasma concentration of medications that include acetaminophen as an ingredient are determined with a conversion algorithm from the determined plasma acetaminophen concentration.

15. A method according to claim 13, wherein the plasma concentration of medications that are coadministered with acetaminophen are determined with a conversion algorithm from the determined plasma acetaminophen concentration.

16. A method according to claim 13 wherein multiple separate CGMs with a consistent and observable distortions due to acetaminophen interference are utilized as (b).

17. A method according to claim 13 wherein the presence of acetaminophen in a patient is detected through the determination of the existence of the distortion effect of acetaminophen on continuous analyte sensors.

18. A method according to claim 13 wherein the presence of medications coadministered with acetaminophen is detected through the determination of the existence of the distortion effect of acetaminophen on continuous analyte sensors.

19. A method according to claim 13 wherein the presence of acetaminophen in a patient is detected through the measurement of the distortion effect of acetaminophen on continuous analyte sensors.

20. A method according to claim 13 wherein the presence of medications coadministered with acetaminophen is detected through the measurement of the distortion effect of acetaminophen on continuous analyte sensors.
